(19) United States
(12) Patent Application Publication
(10) Pub. No.: US 2026/0325808 A1
(43) Pub. Date: Oct. 22, 2026

(54) ELECTRONIC TRANSDERMAL PATCH

(71) Applicant: PHILIP MORRIS PRODUCTS S.A., Neuchatel (CH)

(72) Inventors: Riccardo Riva Reggiori, Lausanne (CH); Christelle Chrea, Signèse (CH)

(21) Appl. No.: 19/106,668

(22) PCT Filed: Aug. 23, 2023
(86) PCT No.: PCT/EP2023/073148

§ 371 Date: Feb. 26, 2025

(30) Foreign Application Priority Data
Aug. 26, 2022 (EP) 22192345.1

(51) Int. Cl. A61M 35/00

(52) U.S. Cl. CPC A61M 35/003; A61M 2202/0007; A61M 2205/10; A61M 2205/12; A61M 2205/3327; A61M 2205/3592; A61M 2205/50; A61M 2210/04; A61M 2230/005; A61M 2230/65

(57) ABSTRACT

The invention relates to an electronic transdermal patch comprising a substrate storage. The substrate storage comprises a first compartment holding a first substrate. The substrate storage comprises a second compartment holding a second substrate. The first substrate is different from the second substrate. The electronic transdermal patch further comprises a sensor. The sensor is configured to detect an amount of the first substrate adjacent the electronic transdermal patch. The electronic transdermal patch further comprises a delivery element. The delivery element is configured to dispense one or both of the first substrate and the second substrate. The electronic transdermal patch further comprises a controller. The controller is configured to control delivery of the first substrate via the delivery element based on a sensor output of the sensor. The invention further relates to a method for delivering substrates with an electronic transdermal patch.

The present invention relates to an electronic transdermal patch. The invention further relates to a method for delivering substrates with an electronic transdermal patch.

A transdermal patch (such as a nicotine patch or other medicated adhesive patch) may be placed on a skin of a user to deliver a dose of a substrate (such as nicotine) through the skin of the user. The transdermal patch generally includes a reservoir to store the substrate. The reservoir may be covered by a porous membrane. During usage, the transdermal patch may release the substrate from the reservoir to the user via the porous membrane. In another example, the reservoir may include a plurality of thin layers that may comprise the substrate. During usage, each layer of the plurality of thin layers may melt based on body heat of the user, to transmit the substrate from the reservoir to the skin of the user.

It would be desirable to have a transdermal patch in which the substrate delivery can be controlled. It would be desirable to have a transdermal patch in which the substrate delivery can be customized. It would be desirable to have a transdermal patch in which the substrate delivery dosage can be controlled. It would be desirable to have a transdermal patch in which at least two substrates can be delivered. It would be desirable to have a transdermal patch in which the delivery of at least two substrates can be controlled.

According to an embodiment of the invention there is provided an electronic transdermal patch comprising a substrate storage. The substrate storage may comprise a first compartment holding a first substrate. The substrate storage may comprise a second compartment holding a second substrate. The first substrate may be different from the second substrate. The electronic transdermal patch may further comprise a sensor. The sensor may be configured to detect an amount of the first substrate adjacent the electronic transdermal patch. The electronic transdermal patch may further comprise a delivery element. The delivery element may be configured to dispense one or both of the first substrate and the second substrate. The electronic transdermal patch may further comprise a controller. The controller may be configured to control delivery of the first substrate via the delivery element based on a sensor output of the sensor.

According to an embodiment of the invention there is provided an electronic transdermal patch comprising a substrate storage. The substrate storage comprises a first compartment holding a first substrate. The substrate storage comprises a second compartment holding a second substrate. The first substrate is different from the second substrate. The electronic transdermal patch further comprises a sensor. The sensor is configured to detect an amount of the first substrate adjacent the electronic transdermal patch. The electronic transdermal patch further comprises a delivery element. The delivery element is configured to dispense one or both of the first substrate and the second substrate. The electronic transdermal patch further comprises a controller. The controller is configured to control delivery of the first substrate via the delivery element based on a sensor output of the sensor.

Detecting an amount of the first substrate adjacent the electronic transdermal patch enables a more optimal delivery of the first substrate. During use of an electronic transdermal patch, the optimal delivery of the first substrate may depend upon a multitude of factors. Particularly, the optimal delivery of the first substrate may depend upon a current first substrate level experienced by a user. Knowledge of this current first substrate level may be gained by measuring the amount of the first substrate adjacent the electronic transdermal patch.

The sensor may be configured to detect an amount of the first substrate on a user's skin adjacent the electronic transdermal patch. The amount of the first substrate on a user skin may be determined by the amount of the first substrate in the sweat of a user. The amount of the first substrate in the sweat of a user may be indicative of the current first substrate level experienced by a user. In other words, by measuring the amount of the first substrate adjacent the electronic transdermal patch, the detector may be able to measure the current first substrate level experienced by a user.

In an example, the sensor of the electronic transdermal patch may determine between 10% and 30%, preferably about 20% first substrate-preferably nicotine-content in the sweat of the user. Based on the determined first substrate content, the electronic transdermal patch may control the delivery element to deliver between 40% and 80%, preferably between 50% and 70%, more preferably about 60% of the first substrate content. As described in more detail below, the measurement is preferably done by the sensor measuring the electrical impedance of the skin of a user.

The delivered first substrate content may thus not exceed the first substrate content that is already present in the body of the user. Overdosing of the first substrate may be avoided.

In an example, the sensor of the electronic transdermal patch may determine between 30% and 50%, preferably about 40% first substrate content in the sweat of the user. Due to a higher first substrate content in comparison to the above example, the electronic transdermal patch may control the delivery element to deliver between 10% and 50%, preferably between 20% and 40%, more preferably about 30% of the first substrate content Again, the delivered first substrate content may thus not exceed the first substrate content that is already present in the body of the user. Overdosing of the first substrate may be avoided.

The electronic transdermal patch may control the delivery of the second substrate—preferably a flavor/fragrance—to be between 10% and 40%, preferably between 20% and 30%, of the second substrate content.

In one example, the electronic transdermal patch may be configured to deliver one or both of the first substrate and the second substrate for a certain period of time. The electronic transdermal patch may deliver a first dosage such as between 2% and 8%, preferably 5% of first substrate content with 0% second substrate content during between 15 min and 45 min, preferably 30 min from the activation of the electronic transdermal patch.

After that, the electronic transdermal patch may deliver a second dosage such as between 10% and 30%, preferably 20% of first substrate content with between 5% and 15%, preferably 10% second substrate content during between 45 min and 75 min, preferably 60 min from the activation of the electronic transdermal patch.

After that, the electronic transdermal patch may deliver a second dosage such as between 30% and 50%, preferably 40% of first substrate content with between 10% and 30%, preferably 20% second substrate content during between 75 min and 105 min, preferably 90 min from the activation of the electronic transdermal patch.

In a similar manner, the electronic transdermal patch may be configured to deliver a fourth dosage, a fifth dosage, a sixth dosage, and the like, based on a user preference.

The electronic transdermal patch may also be configured to deliver one or both of the first substrate and the second substrate at predetermined timeslots. In an example, the predetermined timeslots may correspond to 5-10 minutes after every interval of a predefined time frame, for example between certain minute-based intervals, between certain hour-based intervals, between certain day-based intervals, between certain week-based intervals, or even between certain month-based intervals. Therefore, because of discrete dosages from the electronic transdermal patch, the user may be relieved from the stimulus habituation. It may be understood that the predetermined timeslot (i.e., 5-10 minutes) is merely provided as an example; and the user or the manufacturer of the electronic transdermal patch may modify the predetermined timeslot, based on the user requirements.

The delivery of the second substrate may be an airborne delivery. The second substrate may be delivery to the mouth and/or nose of the user.

The delivery of the second substrate may be configured to mimic a feeling of consumption of an aerosol generating article such as a heat-not-burn article for the user.

The sensor output may then be used by the controller to optimally control the delivery of the first substrate via the delivery element.

The controller may comprise a lookup table comprising an optimal first substrate level experienced by a user. If the sensor output is indicative of a current first substrate level experienced by a user being lower than the optimal first substrate level stored in the lookup table, the controller may increase the amount of first substrate delivered via the delivery element. Likewise, if the sensor output is indicative of a current first substrate level experienced by the user being higher than the optimal first substrate level stored in the lookup table, the controller may decrease the amount of first substrate delivered via the delivery element. The electronic transdermal patch may be configured such that a user can add a desirable profile. The desirable profile may be added to the controller. The desirable profile may comprise a lookup table comprising a desirable optimal first substance level. The desirable profile may be added using a communication interface as described in the following.

Alternatively to storing an optimal first substrate level in a lookup table, the electronic transdermal patch may comprise a communication interface enabling a user to input a desired first substrate level to the controller. The communication interface may comprise one or both of a Wi-Fi communication element and a Bluetooth communication element. A user may be able to input a desired first substrate level to the controller via the communication interface. The user may be able to input the respective information by an external device such as a smartphone, smartwatch, tablet or cloud server. The external device may comprise an app enabling a user to input the respective information.

The first substrate may comprise nicotine. The first substrate may be nicotine.

The second substrate may comprise a flavorant. The second substrate may be a flavorant. The second substrate may comprise or may be caffeine.

Having nicotine as a first substrate may synergistically interact with having caffeine as a second substrate. Typically, a user may desire caffeine after experiencing nicotine or vice versa. The caffeine may be delivered directly after or simultaneously to the delivery of nicotine. Alternatively, the delivery of caffeine may start before the delivery of nicotine. In other words, they may be an overlap between the delivery of nicotine and the delivery of caffeine according to a desired delivery profile of a user.

Exemplarily, the electronic transdermal patch may be configured to deliver the first substrate or the second substrate for between 1 min and 3 min, preferably for 2 min. Subsequently, the electronic transdermal patch may be configured to deliver other of the first substrate or the second substrate for between 4 min and 8 min, preferably for between 5 min and 7 min, more preferably for 6 min. Particularly preferred, the electronic transdermal patch may be configured to deliver caffeine for about 2 min and subsequently to deliver nicotine for about 6 min.

The electronic transdermal patch may be configured to deliver a decreasing amount of one or both of the first substrate and the second substrate over time. In other words, the electronic transdermal patch may be configured to deliver a decreasing concentration of one or both of the first substrate and the second substrate over time. In the above-mentioned example, the electronic transdermal patch may be configured to deliver caffeine for about 2 min with a decreasing caffeine concentration over the 2 min delivery period. Subsequently, the electronic transdermal patch may be configured to deliver nicotine for about 6 min with a decreasing nicotine concentration over the 6 min delivery period.

In a further embodiment, the first substrate may comprise melatonin. The second substrate may in this embodiment comprise an aromatherapy fragrance such as lavender. The synergistic effect between melatonin as a first substrate and lavender as a second substrate may be to help users with sleep disorders or generally to improve the sleep of a user. Delivering melatonin may increase sleep time, shift the phase of sleep, decrease sleep latency and improve sleep efficiency (ratio of sleep time to time in bed). The delivery of lavender as the second substrate may increase the melatonin level in the blood of the user. In order to improve the sleep of the user, the delivery of melatonin may be synergistically combined with the delivery of lavender.

In a further embodiment, the psychoactive properties of culinary spices may be utilized as one or both of the first substrate and the second substrate. Such culinary spices may be dried fruits and seeds such as nutmeg, vanilla, fennel and black pepper, dried flower buds such as cloves, arils such as mace, barks such as cinnamon and Ceylon/cassia, roots and rhizomes such as asarone, ginger, turmeric, galangal and asafetida and stigmas such as saffron. One or both of the first substrate and the second substrate may comprise active ingredients of these spices such as one or more of myristicin, elemicin, safrole, vanillin, piperonal, anethole, piperine, eugenol, coumarin, calamus, gingerols, shogaols, curcumin, 1,8-cineole, β-pinene, resin, gum, essential oil, propenyl-isobutylsulfide, umbelliferone, picrocrocin and safranal. The delivery of such a substrate may have desired effects such as acting as a stimulant, sedative of hallucinogenic. Due to the potential for abuse of these substrates, the controller may be configured to limit the delivery of the amount of these substrates to a safe level.

The first compartment may be fluidly separate from the second compartment. A fluid-impermeable separation wall may be arranged between the first compartment and the second compartment.

This may prevent mixing of the first substrate with the second substrate. This may enable delivery of the first substrate independent of delivery of the second substrate.

In case the electronic transdermal patch is used for delivering a specific drug that has more than two chemical compounds, the substrate storage may be configured to individually store each chemical compound in each compartment.

The delivery element may comprise a first delivery unit which is configured to dispense the first substrate. The delivery element may comprise a second delivery unit which is configured to dispense the second substrate. The first delivery unit may be different from the second delivery unit.

This may enable the controller to separately control delivery of the first substrate via the first delivery unit and delivery of the second substrate via the second delivery unit. Exemplarily, the first delivery unit may be configured to deliver nicotine followed subsequently by the delivery of caffeine by the second delivery unit. The synergistic effect between the delivery of the first substrate and of the second substrate may be enhanced by providing two separate delivery units that can be separately controlled by the controller such that a desired delivery profile of the two substrates can be achieved.

One or both of the first delivery unit and the second delivery unit may be configured as a micro-pump.

One or both of the first delivery unit and the second delivery unit may be configured as a mechanical micropump such as a diaphragm micropump, a piezoelectric micropump and a peristaltic micropump.

Alternatively, one or both of the first delivery unit and the second delivery unit may be configured as a non-mechanical micropump such as a valve-less micropump, a capillary micropump, a chemically powered micropump and a light-powered micropump.

One or both of the first delivery unit and the second delivery unit may be actuated via one or more of a piezoelectric implement, an electrostatic implement, a thermo-pneumatic implement, a pneumatic implement, a magnetic implement, an electro-osmotic implement and an electro-hydrodynamic implement. Alternatively, one or both of the first delivery unit and the second delivery unit may be actuated via one or more of an electrical implement, a hydraulic implement and a mechanical implement.

One or both of the first delivery unit and the second delivery unit may comprise a nozzle such as a sprayer or a heated aerosolizer for delivering one or both of the first substrate and the second substrate.

The electronic transdermal patch according to any of the preceding claims, wherein the delivery of the second substrate via the delivery element is controlled by the controller based on the delivery of the first substrate.

This may be particularly beneficial to synergistically deliver the second substrate as a function of the delivery of the first substrate. As described herein, exemplarily the second substrate may be caffeine and the first substrate may be nicotine. A user may desire caffeine delivery after experiencing nicotine. As a consequence, the controller may control delivery of the second substrate such that the second substrate is delivered immediately after delivery of the first substrate. A further option would be to deliver the second substrate such that an overlap is created between the delivery of the second substrate and the first substrate. In other words, after a predetermined time or a predetermined amount of delivery of the first substrate, the controller may start delivery of the second substrate. The end of the delivery of the first substrate may be after the start of the delivery of the second substrate. Another option would be to wait for a predetermined time with the delivery of the second substrate after the end of the delivery of the first substrate. This may be beneficial if a user desires that the second substrate is delivered after the delivery of the first substrate. As described herein, the controller may comprise a lookup table to determine the optimal delivery profile of the second substrate depending upon the delivery of the first substrate. Alternatively, a user may input the desired delivery profile of the second substrate as a function of the delivery of the first substrate into the controller. This input may be a wireless input such as by a Wi-Fi connection or Bluetooth connection of the controller with an external device such as a smartphone, smartwatch or tablet.

A delivery period of the first substrate via the delivery element may be controlled by the controller based on the sensor output of the sensor. Alternatively or additionally, the rate of delivery of the first substrate via the delivery element may be controlled by the controller based on the sensor output of the sensor. The overall delivery of the first substrate to a user may thus be optimized.

Alternatively or additionally, an amount of first substrate delivered via the delivery element may be controlled by the controller based on the sensor output of the sensor. The benefit may be that an optimal amount of first substrate may be delivered to the user due to the dependence of the delivery upon the sensor output of the sensor.

The delivery element may be configured to deliver the second substate as an airborne delivery. The delivery element may be configured as a nozzle. The delivery element may be configured as a spraying nozzle. If the delivery element comprises a first delivery unit and a second delivery unit as described herein, each of the delivery units may comprise or may be configured as a nozzle or a spraying nozzle.

The sensor may be configured to detect the electrical impedance of the skin of a user adjacent the electronic transdermal patch. The electrical impedance of the skin of a user may be indicative of an amount of first substrate. The electrical impedance of the skin of a user may be indicative of an amount of first substrate in the sweat of a user. The electrical impedance of the skin of a user may be indicative of an amount of first substrate in the bloodstream of a user.

For example, the sensor may include a plurality of microfluidic tubes that have sensing elements. The sensing elements may be configured to measure a concentration of the first substrate content (such as, the nicotine content, a potassium content, a sodium content, metabolites, or a class of molecules that communicate with enzymes) in the sweat of the user. In an example, the sensor may be disposed in the electronic transdermal patch at a location that is proximate and exposed to the skin of the user. The sensor may also include a plurality of electrodes, which may be configured to measure the concentration of the substrate content in the sweat of the user. Examples of the sensor may include an enzyme modified field effect transistor (FET) sensor, a molecularly imprinted polymers (MIP) sensor, a surface plasmon resonance (SPR) sensor, on-skin wearable sweat sensors, near-skin wearable sweat sensors or other bioelectric sensors. Based on the detected substrate, the sensor may be further configured to transmit the detected substrate content to the controller, for the control of the delivery of the first substrate.

One or both of the first compartment and the second compartment may be replaceable or the substrate storage may be replaceable. In other words, one or more of the first compartment, the second compartment and the substrate storage may be configured as a cartridge. The cartridge may be replaced when the respective substrate is depleted.

The electronic transdermal patch may further comprise a communication interface enabling a user to control delivery of one or both of the first substrate and the second substrate via the controller.

The communication interface may enable control of at least one of: an amount of first substrate delivery, an amount of second substrate delivery, a duration of delivery of the first substrate and a duration of delivery of the second substrate.

The controller may be configured to transmit information to an external device such as a smartphone, smartwatch, tablet or a cloud server. The transmitted information that may comprise at least one of: a count of used doses, a count of available doses, a count of used/available first substrate doses, a count of used/available second substrate doses, a count of used flavors/fragrances, a count of available flavors/fragrances, a type of dose (such as nicotine or caffeine), a type of flavor/fragrance (such as a lime flavor or a mint flavor), an over-dosage alert, an under-dosage alert, a level of adhesiveness of an adhesive strip for attaching the electronic transdermal patch to the skin of the user (for example, a low level adhesiveness may indicate that the electronic transdermal patch is about to fall-off because of minimal adhesiveness, and a high level adhesiveness may indicate that the electronic transdermal patch is rigidly coupled to the skin of the user), a dosage timer menu, a dosage selection menu, a flavor/fragrance timer menu, a flavor/fragrance selection menu, and the like. Based on the transmitted information to a graphical user interface of the external device such as a display or touch sensitive display, the delivery profile of the delivery element may be controlled by a user input from the graphical user interface of the external device. Based on the received user input, the controller may be configured to control at least the delivery profile or one or both of the first substrate.

The electronic transdermal patch may further comprise an actuator enabling a user to control delivery of one or both of the first substrate and the second substrate via the controller. The actuator may be configured as a button. The actuator may be configured as a mechanical or electro-mechanical implement such as a knob, slider, lever and the like.

The controller of the electronic transdermal patch may be configured to automatically control delivery of the first substrate based on the sensor output. Particularly, the controller may be configured to automatically control delivery of the nicotine based upon the detected nicotine contact in the sweat of the user.

The controller of the electronic transdermal patch may be configured to automatically end the delivery of the first substrate based on the sensor output or based on an amount of first substrate delivered or based on a first substrate delivery time elapsed.

This may save energy of the electronic transdermal patch. Further, this may ensure that the amount of first substrate delivered does not exceed a predetermined threshold.

The electronic transdermal patch may further comprise an adhesive layer. The adhesive layer may be configured to attach the electronic transdermal patch to the skin of a user.

The electronic transdermal patch may be pad or pouch shaped.

One or more of the substrate storage, the first compartment, the second compartment, the sensor and the controller may be hermetically sealed. The hermetic seal may be provided by a housing of the electronic transdermal patch.

The control of the delivery of one or both of the first substrate and the second substrate by the controller may include control of the delivered total amount of the respective substrate. The control of the delivery of one or both of the first substrate and the second substrate by the controller may include control of the delivered amount over time of the respective substrate. The control of the delivery of one or both of the first substrate and the second substrate by the controller may include control of the time period of delivery of the respective substrate.

The invention further relates to a method for delivering substrates with an electronic transdermal patch as described herein, wherein the method may comprise one or more of the following steps:
detecting, via the sensor, an amount of first substrate, and
controlling, via the controller, delivery of the first substrate via the delivery element based on a sensor output of the sensor.

The method may comprise the step of delivering the second substrate.

The method may comprise the step of delivering the second substrate depending upon the delivery of the first substrate.

The components of the electronic transdermal patch may be provided in a mask or other wearable device such as a wristband or leg-band instead of an electronic transdermal patch. In other words, the invention also relates to a mask or other wearable device such as a wristband or leg-band comprising the herein described components of the electronic transdermal patch. The key concept is applicable in these other applications to measure the amount of first substrate by the sensor and to control the delivery of the first substrate based on the sensor output.

Features described in relation to one embodiment may equally be applied to other embodiments of the invention.

The invention will be further described, by way of example only, with reference to the accompanying drawings in which:

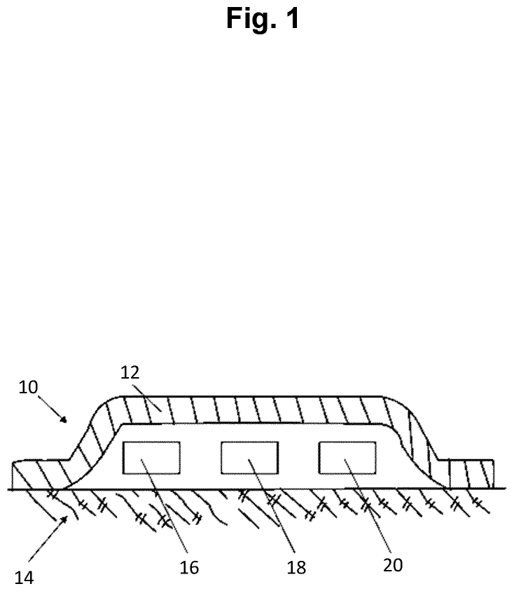

FIG. 1 shows a sectional view of an electronic transdermal patch;

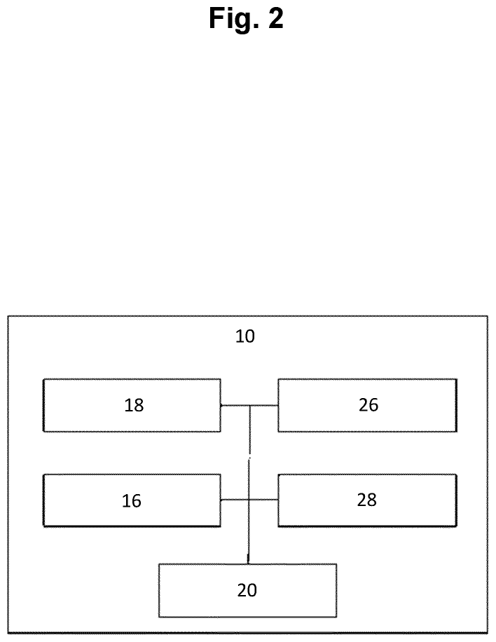

FIG. 2 shows a block diagram of components of the electronic transdermal patch;

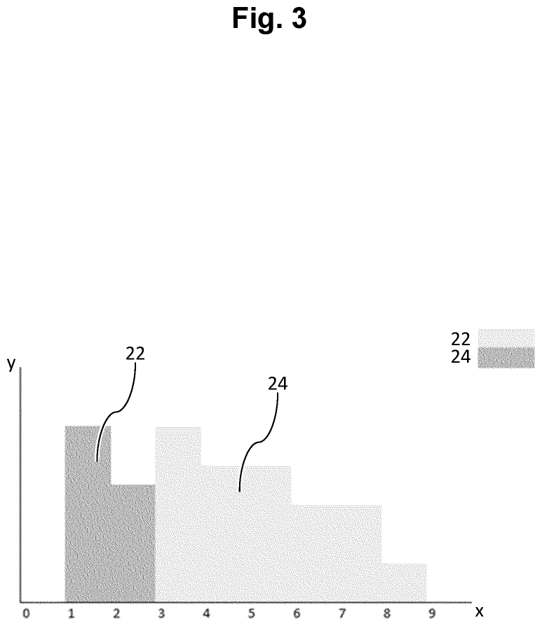

FIG. 3 shows a diagram showing the delivery of first and second substrates over time;

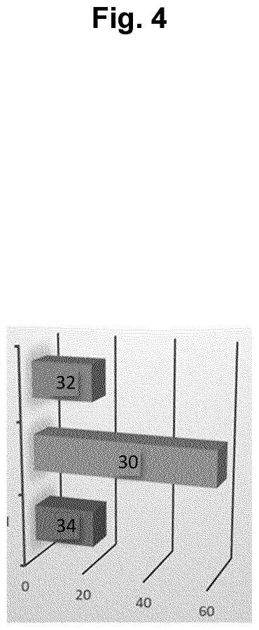

FIG. 4 shows the delivery of first and second substrates depending upon a sensor output;

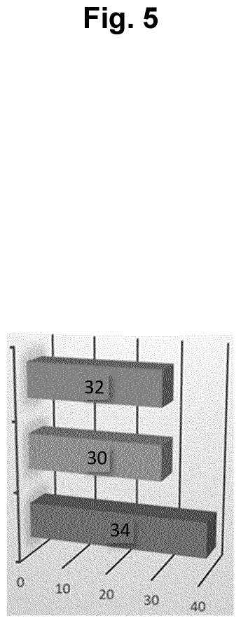

FIG. 5 shows a further example of the delivery of first and second substrates depending upon a sensor output;

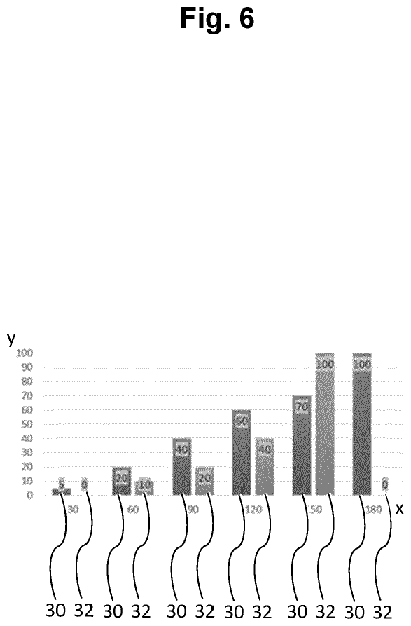

FIG. 6 shows the delivery of first and second substrates;

FIG. 1 shows an electronic transdermal patch 10. The electronic transdermal patch 10 comprises an enclosure 12 or housing. Within the enclosure 12, the components of the transdermal patch are enclosed. The electronic transdermal patch 10 can be attached to the skin 14 of a user. The electronic transdermal patch 10 can be attached to the skin 14 of the user with an adhesive.

The enclosure 12 encloses a substrate storage 16, a sensor 18 and a controller 20. The substrate storage 16 comprises a first compartment (not shown) and a second compartment (not shown). The first compartment is fluidly separated from the second compartment. A first substrate 22 is arranged in the first compartment and a second substrate 24 is arranged in the second compartment. The first substrate 22 is different from the second substrate 24. Preferably, the first substrate 22 is nicotine and the second substrate 24 is caffeine.

FIG. 2 shows a block diagram with further components of the electronic transdermal patch 10. In addition to the substrate storage 16, the sensor 18 and the controller 20, a delivery element comprising a first delivery unit 26 and a second delivery unit 28 are depicted. The first delivery unit 26 is configured to deliver the first substrate 22 and the second delivery unit 28 is configured to deliver the second substrate 24. The first delivery unit 26 may be configured as a nozzle or atomizer or any kind of delivery device. The second delivery unit 28 may be configured similarly. The first delivery unit 26 is separate from the second delivery unit 28. Hence, the first substrate 22 can be delivered independent of the second substrate 24. Delivery of the first substrate 22 and the delivery of the second substrate 24 is controlled by the controller 20. The controller 20 can control delivery of the first substrate 22 independent of the delivery of the second substrate 24.

FIG. 3 shows a delivery profile of the first substrate 22 and of the second substrate 24. The X axis shows the time in minutes and the Y axis shows the amount of substrate delivered. In the example of FIG. 3, the first substrate 22 is delivered initially. Starting at minute 1, the first substrate 22 is delivered for two minutes. During the time of delivery of the first substrate 22, the amount of first substrate 22 delivered over time is reduced. After minute 3, delivery of first substrate 22 is stopped. Directly after stopping the delivery of the first substrate 22, the second substrate 24 is delivered. The second substrate 24 is delivered over a period of around six minutes. During that time, the amount of second substrate 24 delivered is reduced over time.

FIG. 4 shows an example of the functionality of the sensor 18 of the electronic transdermal patch 10. In more detail, the first substrate content 30, meaning the amount of first substrate 22 delivered over a predetermined time, depends upon the measurement of the sensor 18. The sensor 18 is arranged to measure the electrical impedance of the skin 14 of the user. The electrical impedance of the skin 14 of a user is indicative of the amount of first substrate 22 contained in the sweat of a user which in turn is indicative of the amount of first substrate 22 in the bloodstream of the user. In other words, the sensor 18 is configured to measure the first substrate 22 concentration the user is currently experiencing. As a function of the first substrate measurement 34 of the sensor 18, the controller 20 is configured to control delivery of the first substrate content 30. In the example shown in FIG. 4, a nicotine content of 20% (first substrate measurement 34) is detected by the sensor 18. As a consequence, a relatively high nicotine content of 60% (first substrate content 30) is delivered. Further, FIG. 4 shows a delivery of the second substrate 24 in the form of a second substrate content 32 being 20% of caffeine.

FIG. 5 shows an example similar to the example shown in FIG. 4 with the difference that a higher nicotine content of 40% (first substrate measurement 34) is detected by the sensor 18. As a consequence, a lower nicotine content of 30% (first substrate content 30) is delivered. FIG. 5 also shows that the second substrate content 32 may be varied. In the example shown in FIG. 5, a second substrate content 32 of 30% caffeine is delivered.

FIG. 6 shows an exemplary delivery profile of nicotine and caffeine over time. The X axis again shows the time in minutes and the Y axis shows the amount of substrate delivered. After a time of 30 minutes after activation of the electronic transdermal patch 10, a first dosage (first substrate content 30) of 5% nicotine is delivered while—at that stage—no second substrate 24 is delivered. After a time of 60 minutes after activation of the electronic transdermal patch 10, a second dosage of 20% nicotine is delivered together with a first dosage of 10% caffeine. After a time of 90 minutes after activation of the electronic transdermal patch 10, a third dosage of 40% nicotine is delivered together with a second dosage of 20% caffeine. After a time of 120 minutes after activation of the electronic transdermal patch 10, a fourth dosage of 60% nicotine is delivered together with a third dosage of 40% caffeine. After a time of 150 minutes after activation of the electronic transdermal patch 10, a user may want to experience a higher content of caffeine and a fifth dosage of 70% nicotine is delivered together with a fourth dosage of 100% caffeine. Finally, after a time of 180 minutes after activation of the electronic transdermal patch 10, a sixth dosage of 100% nicotine is delivered while-at that stage-no second substrate 24 is delivered.

1-15. (canceled)

16. An electronic transdermal patch comprising:
a substrate storage, wherein the substrate storage comprises a first compartment holding a first substrate, wherein the substrate storage comprises a second compartment holding a second substrate, wherein the first substrate is different from the second substrate,
a sensor, wherein the sensor is configured to detect an amount of the first substrate adjacent the electronic transdermal patch,
a delivery element, wherein the delivery element is configured to dispense one or both of the first substrate and the second substrate,
a controller, wherein the controller is configured to control delivery of the first substrate via the delivery element based on a sensor output of the sensor or based on a predefined profile,
wherein the sensor is configured to detect an amount of the first substrate on a user's skin adjacent the electronic transdermal patch
wherein the second substrate comprises a flavorant,
wherein the delivery element is configured to deliver the second substate as an airborne delivery.

17. The electronic transdermal patch according to claim 16, wherein the first substrate comprises nicotine, preferably wherein the first substrate is nicotine.

18. The electronic transdermal patch according to claim 16, wherein the second substrate is a flavorant, preferably wherein the second substrate comprises or is caffeine.

19. The electronic transdermal patch according to claim 16, wherein the first compartment is fluidly separate from the second compartment, preferably wherein a fluid-impermeable separation wall is arranged between the first compartment and the second compartment.

20. The electronic transdermal patch according to claim 16, wherein the delivery element comprises a first delivery unit which is configured to dispense the first substrate, wherein the delivery element comprises a second delivery unit which is configured to dispense the second substrate, wherein the first delivery unit is different from the second delivery unit., preferably wherein one or both of the first delivery unit and the second delivery unit is configured as a micro-pump.

21. The electronic transdermal patch according to claim 16, wherein the delivery of the second substrate via the delivery element is controlled by the controller based on the delivery of the first substrate.

22. The electronic transdermal patch according to claim 16, wherein a delivery period of the first substrate via the delivery element is controlled by the controller based on the sensor output of the sensor.

23. The electronic transdermal patch according to claim 16, wherein a delivered substrate amount of the first substrate via the delivery element is controlled by the controller based on the sensor output of the sensor.

24. The electronic transdermal patch according to claim 16, wherein the sensor is configured to detect the electrical impedance of the skin of a user adjacent the electronic transdermal patch.

25. The electronic transdermal patch according to claim 16, wherein one or both of the first compartment and the second compartment is replaceable or wherein the substrate storage is replaceable.

26. The electronic transdermal patch according to claim 16, wherein the electronic transdermal patch further comprises a communication interface enabling a user to control delivery of one or both of the first substrate and the second substrate via the controller.

27. The electronic transdermal patch according to claim 16, wherein the electronic transdermal patch further comprises an actuator enabling a user to control delivery of one or both of the first substrate and the second substrate via the controller.

28. A method for delivering substrates with an electronic transdermal patch according to claim 16, wherein the method comprises:
detecting, via the sensor, an amount of first substrate, and
controlling, via the controller, delivery of the first substrate via the delivery element based on a sensor output of the sensor.
